(19) United States
(12) Patent Application Publication
(10) Pub. No.: US 2012/0130200 A1
(43) Pub. Date: May 24, 2012

(54) SYSTEM AND METHOD FOR AUTOMATED INFANT ASSESSMENT

(75) Inventor: James Patrick Cipriano, Laurel, MD (US)

(73) Assignee: GENERAL ELECTRIC COMPANY, Schenectady, NY (US)

(21) Appl. No.: 12/949,282

(22) Filed: Nov. 18, 2010

(51) Int. Cl. A61B 5/00

(52) U.S. Cl. 600/301

(57) ABSTRACT

A system for automatically assessing an infant includes a computer. The computer is configured to assess an infant's appearance and generate an appearance score based on the appearance assessment, assess the infant's pulse and generate a pulse score based on the pulse assessment, assess the infant's reaction to a stimulus and generate a grimace score based on the assessment of the infant's reaction to the stimulus, assess the infant's activity, and generate an activity score based on the activity assessment, and assess the infant's respiration and generate a respiration score based on the respiration assessment. The computer is also configured to generate a composite infant assessment based on the appearance score, the pulse score, the grimace score, the activity score and the respiration score.

### BACKGROUND OF THE INVENTION

The subject matter disclosed herein relates to a system and method for the automated assessment of an infant. More precisely, the subject matter relates to a system and method for automatically conducting an APGAR test.

The APGAR test has traditionally comprised a manually performed test adapted to assess the health and general well being of an infant. The APGAR test includes the evaluation of the following five criteria: Appearance; Pulse: Grimace; Activity and Respiration. Each criterion is typically manually assessed and assigned a score of 0-2. The individual criterion scores are summed to form a composite APGAR score in the range of 0-10. APGAR scores of 7-10 are generally considered normal, and APGAR scores below 7 often indicate an area of concern.

One problem with conventional APGAR tests relates to the subjectivity inherent in the manual assessment of the individual APGAR criteria. This subjectivity can yield imprecision in the resultant APGAR score. Another problem with conventional APGAR tests relates to the time and labor required to manually assess the individual APGAR criteria.

### BRIEF DESCRIPTION OF THE INVENTION

The above-mentioned shortcomings, disadvantages and problems are addressed herein which will be understood by reading and understanding the following specification.

In an embodiment, a method for automatically assessing an infant includes implementing a computer to assess the infant's appearance and to generate an appearance score based on the appearance assessment. The method also includes implementing the computer to assess the infant's pulse and to generate a pulse score based on the pulse assessment. The method also includes implementing the computer to assess the infant's reaction to a stimulus and to generate a grimace score based on the assessment of the infant's reaction to the stimulus. The method also includes implementing the computer to assess the infant's activity and to generate an activity score based on the activity assessment. The method also includes implementing the computer to assess the infant's respiration and to generate a respiration score based on the respiration assessment. The method also includes implementing the computer to generate a composite infant assessment based on the appearance score, the pulse score, the grimace score, the activity score and the respiration score.

In another embodiment, a system for automatically assessing an infant includes a computer. The computer is configured to assess an infant's appearance and generate an appearance score based on the appearance assessment, assess the infant's pulse and generate a pulse score based on the pulse assessment, assess the infant's reaction to a stimulus and generate a grimace score based on the assessment of the infant's reaction to the stimulus, assess the infant's activity and generate an activity score based on the activity assessment, and assess the infant's respiration and generate a respiration score based on the respiration assessment. The computer is also configured to generate a composite infant assessment based on the appearance score, the pulse score, the grimace score, the activity score and the respiration score.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of an automated infant assessment system in accordance with an embodiment; and

FIG. 2 is a flow chart illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

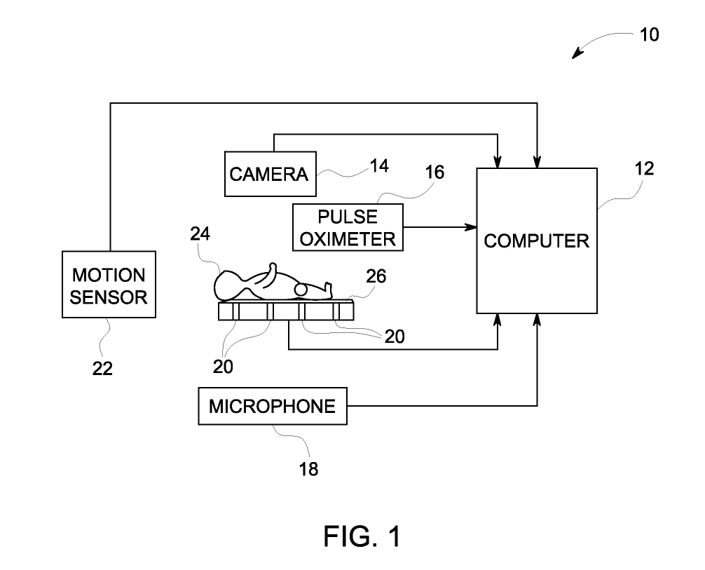

Referring to FIG. 1, a schematic representation of an automated infant assessment system 10 is shown in accordance with an embodiment. The system 10 may comprise a computer 12, a camera 14, a pulse oximeter 16; a microphone 18; one or more pressure sensors 20; and a motion sensor 22.

The computer 12 is operatively connected to and adapted to receive data from each of the camera 14, the pulse oximeter 16; the microphone 18; the pressure sensors 20; and the motion sensor 22. According to one embodiment, the camera 14 comprises a digital camera adapted to generate digital images of an infant 24, and to transmit the digital images to the computer 12. The digital images from the camera 14 may comprise still shots and/or full motion video. The computer 12 may implement the still shots to assess the appearance of the infant, and the full motion to track and assess infant motion.

The pulse oximeter 16 may comprise a sensor placed across the foot of the infant 24. The pulse oximeter 16 is adapted to monitor the oxygenation of the infant's hemoglobin in a known manner, and to transfer the oxygenation data to the computer 12.

The microphone 18 is adapted to record sound from the infant 18, and to transmit the recorded sound data to the computer 12. According to one embodiment, the pressure sensors 20 may comprise an array of uniformly arranged sensors 20 disposed within or beneath an infant mattress 26. Each sensor 20 within the array can be configured to measure pressure or load generated by the mass of an infant supported by the mattress 26, and to transmit the recorded pressure data to the computer 12. The computer 12 may be adapted to estimate the degree and type of infant motion based on feedback from the sensors 20. As an example, the computer 12 may be configured to individually assess feedback from each sensor 20 within the array, define the pressure distribution across the surface of the mattress 26 based on feedback from the sensors 20, and estimate the degree and type of infant motion based on the pressure distribution.

The motion sensor 22 may comprise any known technology adapted to monitor the motion of the infant 24, and to transmit the recorded motion data to the computer 12. In a non-limiting manner, the motion sensor 22 may comprise a mechanical device, an electrical device, an optical device, an acoustic device, or a magnetic device configured to monitor motion in a known manner.

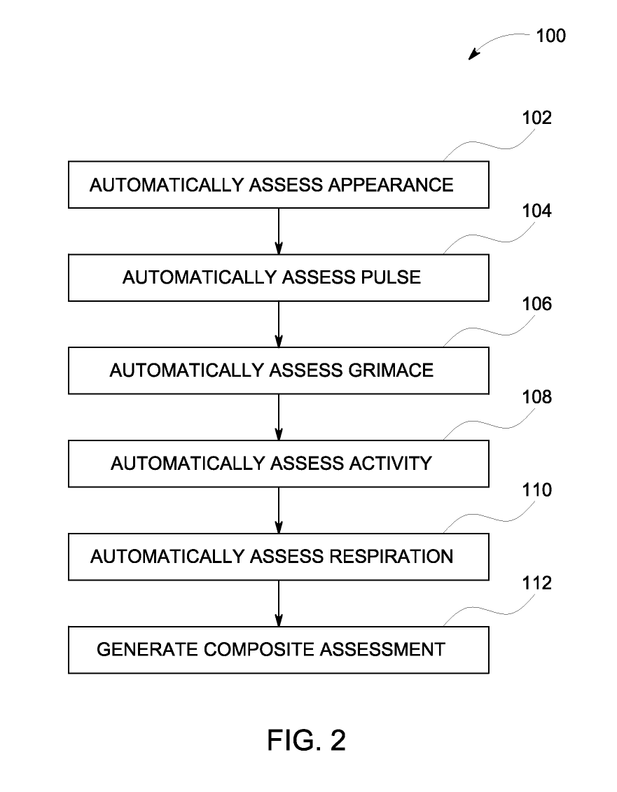

Having described exemplary components of the system 10, a method for implementing the system 10 to automatically assess an infant will now be described. Referring to FIG. 2, a flow chart illustrating an algorithm 100 is shown in accordance with an embodiment. The technical effect of the algorithm 100 is to automatically assess the health of an infant. According to one embodiment, the at least a portion of the algorithm 100 comprises a computer program stored on a computer-readable storage medium of the computer 12 (shown in FIG. 1). The individual blocks 102-112 represent steps that can be performed by the computer 12. Steps 102-112 need not necessarily be performed in the order shown.

The algorithm 100 is adapted to automatically assess an infant based on the APGAR test criteria. The APGAR criteria include Appearance, Pulse, Grimace, Activity and Respiration. It should be appreciated that the automation of the infant assessment reduces subjectivity associated with a manual assessment, and also minimizes labor requirements.

Referring to FIGS. 1 and 2, at step 102 the algorithm 100 automatically assess the infant's appearance. The appearance assessment of step 102 may be acquired in accordance with a first embodiment based on the infant's skin color. For example, the camera 14 may acquire a digital photograph of the infant 12, and transmit the digital photograph to the computer 12 for automated assessment. The computer 12 may comprise software adapted to automatically assess the color spectrum of the infant's trunk and extremities. The computer 12 could generate a low appearance score (e.g., 0) if, based on the infant photograph, the infant does not include any pink coloration such that its trunk and extremities remain generally blue. The computer 12 could generate an intermediate appearance score (e.g., 1) if the infant's trunk comprises pink coloration while the extremities remain generally blue. The computer 12 could generate a high appearance score (e.g., 2) if the infant's trunk and extremities are both generally pink.

The appearance assessment of step 102 may be acquired in accordance with a second embodiment based on the oxygenation of the infant's hemoglobin as measured by the pulse oximeter 16. It is well known that deoxygenated hemoglobin is indicative of cyanosis, which is a blue coloration of the skin and mucous membranes. It is also well known that oxygen saturation is indicative of acrocyanosis, which is a blue coloration of the extremities. Accordingly, the computer 12 could generate a low appearance score (e.g., 0) based on pulse oximeter data indicating deoxygenated hemoglobin, an intermediate appearance score (e.g., 1) based on pulse oximeter data indicating oxygen saturation of the infant's hemoglobin, and a high appearance score (e.g., 2) based on normal oxygenation data from the pulse oximeter 16.

At step 104 the algorithm 100 automatically assesses the infant's pulse. According to one embodiment, the pulse oximter 16 could measure the infant's pulse and transmit the measured pulse data to the computer 12. The computer 12 could generate a low pulse score (e.g., 0) if no pulse is detected. The computer 12 could generate an intermediate pulse score (e.g., 1) if the measured pulse is less than 100 beats per minute. The computer 12 could generate a high pulse score (e.g., 2) if the measured pulse is equal to or greater than 100 beats per minute.

At step 106 the algorithm 100 automatically assesses the infant's grimace. The grimace assessment should be defined broadly for purposes of this disclosure as an assessment of the infant's reaction to a stimulus. The stimulus may be manually performed or automated and may, for example, comprise a foot strike.

The grimace assessment of step 106 may be acquired in accordance with a first embodiment based on an evaluation of the infant's facial expression in response to a predefined stimulus. After stimulating the infant, the camera 14 may acquire a digital photograph of the infant's face and transmit the digital photograph to the computer 12 for automated assessment. The computer 12 may comprise facial recognition software adapted to automatically assess facial contortion and the degree to which the infant grimaces. The computer 12 could generate a low grimace score (e.g., 0) if, based on the infant photograph, the infant's face does not contort or grimace. The computer 12 could generate an intermediate grimace score (e.g., 1) if the infant's face only minimally contorts or grimaces. The computer 12 could generate a high grimace score (e.g., 2) if the infant's face significantly contorts or grimaces.

The grimace assessment of step 106 may be acquired in accordance with a second embodiment based on an evaluation of the infant's vocal expression in response to a predefined stimulus. After stimulating the infant, the microphone 18 may record the infant's vocal expression and transmit the vocal data to the computer 12 for automated assessment. The computer 12 may comprise software adapted to automatically assess the volume and/or frequency of the recorded vocal data. The computer 12 could generate a low grimace score (e.g., 0) if there is no vocal response. The computer 12 could generate an intermediate grimace score (e.g., 1) if the recorded vocal response is below a predefined volume and/or within a first predefined frequency range. The computer 12 could generate a high grimace score (e.g., 2) if the recorded vocal response is at or above the predefined volume and/or within a second predefined frequency range.

The grimace assessment of step 106 may be acquired in accordance with a third embodiment based on an evaluation of the infant's motion in response to a predefined stimulus. After stimulating the infant the camera 14, the pressure sensors 20 and/or the motion sensor 22 may be implemented to record infant motion and transmit the motion data to the computer 12 for automated assessment. The computer 12 could generate a low grimace score (e.g., 0) if there is no infant motion. The computer 12 could generate an intermediate grimace score (e.g., 1) if there is moderate infant motion. The computer 12 could generate a high grimace score (e.g., 2) if there is extensive infant motion.

At step 108 the algorithm 100 automatically assesses the infant's activity. The activity assessment of the infant 12 may be acquired by implementing the camera 14, the motion sensor 22 and/or pressure sensors 20 to detect the degree of infant motion or infant muscle tone. The computer 12 could generate a low activity score (e.g., 0) if there is no infant motion. The computer 12 could generate an intermediate activity score (e.g., 1) if there is visible muscle tone variation (e.g., muscle flex) and/or minimal infant motion. The computer 12 could generate a high activity score (e.g., 2) if there is significant infant motion.

At step 110 the algorithm 100 automatically assesses the infant's respiration. The respiration assessment of the infant 12 may be acquired in accordance with a first embodiment by implementing the camera 14, the motion sensor 22 and/or pressure sensors 20 to monitor the infant's motion. As an example, the camera 14, the motion sensor 22 and/or pressure sensors 20 may be implemented to monitor a sequence of chest compressions and/or expansions. The computer 12 could generate a low respiration score (e.g., 0) if there are no detectable chest compressions or expansions. The computer 12 could generate an intermediate respiration score (e.g., 1) if the chest compressions and expansions are irregular or weak (e.g., minimally detectable). The computer 12 could generate a high respiration score (e.g., 2) if the chest compressions and expansions are regular and strong.

The respiration assessment of step 110 may be acquired in accordance with a second embodiment based on the infant's vocal expression. It has been observed that a substantial vocal expression (e.g., loud cry) is indicative of healthy respiration such that the respiration assessment may rely on the degree to which an infant is vocal. The microphone 18 may be implemented to record the infant's vocal expression and transmit the vocal data to the computer 12 for automated assessment. The computer 12 may comprise software adapted to automatically assess the volume and/or frequency of the recorded vocal data. The computer 12 could generate a low respiration score (e.g., 0) if the infant 24 is silent. The computer 12 could generate an intermediate respiration score (e.g., 1) in response to a weak cry or audible indications that the infant is gasping. The computer 12 could generate a high respiration score (e.g., 2) in response to a loud cry.

At step 112 the algorithm 100 automatically generates a composite infant assessment based on the appearance score, the pulse score, the grimace score, the activity score and the respiration score. According to one embodiment the computer 12 adds the individual scores, which each comprise a range of 0-2, to generate a composite APGAR score in the range of 0-10. An APGAR score of 7-10 may be considered normal, while a score below 7 could indicate an area of concern.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. A method for automatically assessing an infant comprising:
implementing a computer to assess the infant's appearance, and to generate an appearance score based on the assessment of the infant's appearance;
implementing the computer to assess the infant's pulse, and to generate a pulse score based on the assessment of the infant's pulse;
implementing the computer to assess the infant's reaction to a stimulus, and to generate a grimace score based on the assessment of the infant's reaction to the stimulus;
implementing the computer to assess the infant's activity, and to generate an activity score based on the assessment of the infant's activity;
implementing the computer to assess the infant's respiration, and to generate a respiration score based on the assessment of the infant's respiration; and
implementing the computer to generate a composite infant assessment based on the appearance score, the pulse score, the grimace score, the activity score and the respiration score.

2. The method of claim 1, wherein said implementing the computer to assess the infant's appearance comprises implementing the computer to assess the infant's skin color.

3. The method of claim 1, wherein said implementing the computer to assess the infant's appearance comprises implementing the computer to assess the oxygenation of the infant's hemoglobin based on feedback from a pulse oximeter.

4. The method of claim 1, wherein said implementing the computer to assess the infant's reaction to a stimulus comprises implementing the computer to assess the infant's facial expression.

5. The method of claim 1, wherein said implementing the computer to assess the infant's reaction to the stimulus comprises implementing the computer to assess the infant's vocal expression.

6. The method of claim 1, wherein said implementing the computer to assess the infant's reaction to the stimulus comprises implementing the computer to assess the infant's motion.

7. The method of claim 1, wherein said implementing the computer to assess the infant's activity comprises implementing the computer to assess the infant's motion.

8. The method of claim 1, wherein said implementing the computer to assess the infant's respiration comprises implementing the computer to assess the infant's motion.

9. The method of claim 8, wherein said implementing the computer to assess the infant's motion comprises implementing the computer to assess a sequence of chest contractions and/or expansions.

10. A system for automatically assessing an infant comprising:
a computer configured to:
assess an infant's appearance, and to generate an appearance score based on the assessment of the infant's appearance;
assess the infant's pulse, and to generate a pulse score based on the assessment of the infant's pulse;
assess the infant's reaction to a stimulus, and to generate a grimace score based on the assessment of the infant's reaction to the stimulus;
assess the infant's activity, and to generate an activity score based on the assessment of the infant's activity;
assess the infant's respiration, and to generate a respiration score based on the assessment of the infant's respiration; and
generate a composite infant assessment based on the appearance score, the pulse score, the grimace score, the activity score and the respiration score.

11. The system of claim 10, further comprising a camera, wherein said computer is configured to assess the infant's appearance based on feedback from the camera.

12. The system of claim 11, wherein said computer is configured to assess the infant's reaction to a stimulus based on feedback from the camera.

13. The system of claim 10, further comprising a pulse oximeter, wherein said computer is configured to assess the infant's pulse based on feedback from the pulse oximeter.

14. The system of claim 10, further comprising a microphone, wherein said computer is configured to assess the infant's pulse based on feedback from the microphone.

15. The system of claim 14, wherein said computer is configured to assess the infant's reaction to a stimulus based on feedback from the microphone.

16. The system of claim 10, further comprising a motion sensor, wherein said computer is configured to assess the infant's activity based on feedback from the motion sensor.

17. The system of claim 16, wherein said computer is configured to assess the infant's reaction to a stimulus based on feedback from the motion sensor.

18. The system of claim 10, further comprising a pressure sensor, wherein said computer is configured to assess the infant's activity based on feedback from the pressure sensor.

19. The system of claim 18, wherein said computer is configured to assess the infant's reaction to a stimulus based on feedback from the pressure sensor.
